(19) United States
(12) Patent Application Publication
(10) Pub. No.: US 2015/0011424 A1
(43) Pub. Date: Jan. 8, 2015

(54) METHOD FOR DETERMINING LIVER FAT AMOUNT AND METHOD FOR DIAGNOSING NAFLD

(71) Applicant: Teknologian Tutkimuskeskus VTT, VTT (FI)

(72) Inventors: Matej Oresic, Espoo (FI); Tuulia Hyötyläinen, Espoo (FI); Gopalacharyulu Peddinti, Helsinki (FI); Sandra Castillo, Espoo (FI); Hannele Yki-Järvinen, Espoo (FI)

(21) Appl. No.: 14/372,895

(22) PCT Filed: Jan. 30, 2013
(86) PCT No.: PCT/FI2013/050096

§ 371 Date: Jul. 17, 2014

Related U.S. Application Data
(60) Provisional application No. 61/592,744, filed on Jan. 31, 2012.

(30) Foreign Application Priority Data
Jan. 31, 2012 (FI) 20125103

(51) Int. Cl. G01N 33/49; G01N 30/72

(52) U.S. Cl. CPC G01N 33/492; G01N 30/7233; G01N 2570/00; G01N 2800/085; G01N 2405/02; G01N 2405/04; G01N 2405/08
USPC 506/12; 250/282

(57) ABSTRACT

The present invention is based on the idea of determining certain molecular lipids from a subject's blood sample, for example from serum or plasma sample, and based on the amounts of the determined lipids determining the amount of liver fat and/or diagnosing NAFLD in the subject. More specifically the subjects with elevated liver fat amount and NAFLD are characterized by elevated triglycerides with low carbon number and double bond content in the blood sample. Lysophosphatidylcholines, ether phospholipids, sphingomyelins and PUFA-containing phospholipids are diminished in the blood samples of subjects with an elevated liver fat amount and NAFLD. The method of the present invention can be further used for monitoring the subject's response to the treatment of NAFLD or to the treatment of lowering of the liver fat amount in the subject.

### FIELD OF THE INVENTION

The present invention relates to a method for determining liver fat amount and to a method for diagnosing or monitoring non-alcoholic fatty liver disease.

### BACKGROUND OF THE INVENTION

Non-alcoholic fatty liver disease (NAFLD) is defined as hepatic fat accumulation exceeding 5-10% of liver weight in the absence of other causes of steatosis. NAFLD covers a spectrum of liver abnormalities ranging from simple steatosis to non-alcoholic steatohepatitis (NASH), fibrosis, and cirrhosis¹. In Finland, a population based study showed that the prevalence of NAFLD is 21% in adults aged 45-60 years. NAFLD both precedes and predicts type 2 diabetes, the metabolic syndrome, and cardiovascular disease. NAFLD has thus been predicted to become the number one indication of liver transplantation by the year 2020. It is therefore highly desirable to develop easily available diagnostic tools which would help identifying subjects with a fatty liver.

Liver fat content can be quantitatively measured invasively by a liver biopsy and non-invasively by proton magnetic resonance spectroscopy (¹H-MRS), but these tools are often unavailable in clinical practice. All other radiological techniques provide qualitative rather than quantitative estimates, and their sensitivity is limited. Elevated liver function tests alone are both unspecific and insensitive markers of a fatty liver.

Publication WO 2008/021192 discloses a method for diagnosing or monitoring a liver disorder in a subject by determining the amount of one or more lipid metabolites in one or more samples from a body fluid of the subject and correlating the amounts of the lipid metabolites with the presence of the liver disorder. The lipid metabolites are fatty acids and/or eicosanoids. The liver disorder is hepatic impairment, hepatic steatosis, non-alcoholic fatty liver disease (NAFLD), steatohepatitis or non-alcoholic steatohepatitis (NASH).

Kalhan et al have disclosed in “Plasma metabolomics profile in non-alcoholic fatty liver disease”, Metabolism, Clinical and Experimental (2011), 60(3), 404-413, an examination of the plasma profile of subjects with non-alcoholic fatty liver disease (NAFLD), steatosis and steatohepatitis (NASH) by using an untargeted global metabolomics analysis. The metabolomics analysis showed markedly higher levels of glycocholate, taurocholate and glycochnodeoxychoate in subjects with NAFLD. Plasma concentrations of long chain fatty acids were lower and concentrations of free carnitine, butyrylcarnitine, and methylbutyryl carnitine were higher in NASH. Several glutamyldipeptides were higher whereas cysteine-glutathione levels were lower in NASH and steatosis. Plasma metabolomics analysis revealed marked changes in bile salts and in biochemical related to gluthatione in subjects with NAFLD.

Puri et al, The Plasma Lipidomic Signature of Non-alcoholic Steatohepatitis, Hepatology (Hoboken, N.J., United States) (2009), 50(6), 1827-1838, discloses an analysis of plasma lipids and eicosanoid metabolites quantified by mass spectrometry in NAFL (non-alcoholic fatty liver) and NASH subjects and compared with normal controls. The key findings include significantly increased total plasma monounsaturated fatty acids driven by palmitoleic (16:1 n7) and oleic (18:1 n9) acids content. The levels of palmitoleic acid, oleic acid and palmitoleic acid to palmitic acid (16:0) ratio were significantly increased in NAFLD across multiple lipid classes.

Puri et al, A Lipidomic Analysis of Nonalcoholic Fatty Liver Disease, Hepatology (Hoboken, N.J., United States) (2007), 46(4), 1081-1090, discloses that the mean (nmol/g of tissue) diacylglycerol and triacylglycerol increased significantly in NAFLD, but free fatty acid remained unaltered. The publication also relates to a study, wherein the free fatty acid, diacylglycerol, triacylglycerol, free cholesterol, cholesterol ester and phospholipid contents in normal livers were quantified and compared to those of NAFL (non-alcoholic fatty liver) and NASH, and the distribution of fatty acids within these classes was compared across these groups.

WO 2011/036117 relates to methods for the diagnosis of non-alcoholic steatosis (NASH). The method relies on the determination of certain metabolic markers in a biological sample of the patient which are up- or down-regulated in the NASH patients vs. patients with a simple fatty liver (steatosis). The publication does not relate to the determination of liver fat amount.

WO 2010/018165 relates to methods for the diagnosing of non-alcoholic steatosis (NASH). The method relies on the determination of certain metabolic markers in a biological sample of the patient which are up- or down-regulated in the NASH patients vs. patients with a simple fatty liver (stetosis). The methods cannot be used for determining liver fat amount or NAFLD.

WO 2009/059150 discloses diagnosing whether a subject has steatohepatitis comprising analyzing a biological sample from a subject to determine the levels of one or more biomarkers for steatohepatitis in the sample, where the biomarkers given in the specification are, eg. Metabolite-3073, glutamate, isocitrate, isoleucine, Metabolite-5769, Metabolite-11728, leucine, Metabolite-4274, glycocholate, glutamylvaline, alanine, tyrosine or Metabolite-10026; and comparing the levels of the biomarkers in the sample to steatohepatitis-positive and/or steatohepatitis-negative reference levels of the biomarkers in order to diagnose whether the subject has steatohepatitis. The publication refers to diagnosing NASH not to diagnosing NAFLD or determining the amount of liver fat.

There is clearly a strong need for a specific, accurate, easy to perform and cost effective way for determining a subject's liver fat amount and for diagnosing NAFLD. This enables detecting an increase in the amount of liver fat at an early stage, and also diagnosing NAFLD at an early stage before the disease has exacerbated.

### BRIEF DESCRIPTION OF THE INVENTION

An object of this invention is to provide a specific, accurate, easy to perform and cost effective way for determining a subject's liver fat amount and for diagnosing NAFLD. The present invention is based on the idea of determining certain molecular lipids from a subject's blood sample and based on the amounts of the determined lipids determining the amount of liver fat and/or diagnosing NAFLD in the subject. The present invention provides a method for determining a subject's liver fat amount, which method comprises providing a blood sample from said subject, determining the concentration of at least one molecular lipid in the blood sample, wherein the molecular lipid is selected from
i) group A consisting of TG(16:0/16:0/18:1), TG(16:0/18:0/18:1), TG(16:0/16:0/16:0) and TG(16:0/18:1/18:1), and/or
ii) group B consisting of lysoPC(16:0), lysoPC(18:0), SM(d18:0/18:0), SM(d18:1/24:1), SM(d18:1/16:0), PC(34:2), PC(18:1/22:6), PC(O-24:1/20:4), PC(34:1e), PC(34:2p), PE(38:2) and PE(36:2),

wherein an increased concentration of at least one molecular lipid of group A compared to the normal mean concentration in healthy individuals without NAFLD of respective molecular lipid, and/or

a decreased concentration of at least one molecular lipid of group B compared to the normal mean concentration in healthy individuals without NAFLD of respective molecular lipid correlates with the liver fat amount in said subject.

The present invention also provides a method for diagnosing non-alcoholic fatty liver disease (NAFLD) and/or monitoring subject's response to the treatment or prevention of non-alcoholic fatty liver disease (NAFLD). Object of the invention is achieved by a method characterized by what is stated in the independent claim. Preferred embodiments of the invention are disclosed in the dependent claims.

An advantage of the method of the invention is that it provides a simple and sensitive, non-invasive tool for determining the amount of liver fat and a method for diagnosing NAFLD in a subject.

### ABBREVIATIONS USED IN THIS APPLICATION

¹H-MRS proton magnetic resonance spectroscopy
ALT alanine aminotransferase
ALP alkaline phosphatase
AST aspartate aminotransferase
AUC area under the (ROC) curve
BIC Bayesian information criterion
BMI body mass index
fS fasting serum
GGT gamma-glutamyltransferase
HbA_{1c} glycosylated hemoglobin A_{1c}
HDL high-density lipoprotein
IA intra-abdominal
LDL low-density lipoprotein
LFAT amount of liver fat, percentage of liver tissue weight
LFT liver function tests
lysoPC lysophosphatidylcholine
MRI magnetic resonance imaging
MUFA monounsaturated fatty acid
NAFL non-alcoholic fatty liver
NAFLD non-alcoholic fatty liver disease
NASH non-alcoholic steatohepatitis
NS non-significant
OGTT oral glucose tolerance test
PC phosphatidylcholine
PUFA polyunsaturated fatty acid
ROC receiver operator statistics
SC subcutaneous
SFA saturated fatty acid
siRNA small interfering RNA
SM sphingomyelin
SNP single nucleotide polymorphism
SREBP sterol regulatory element binding protein
TG triglyceride
TLC thin-layer chromatography
VLDL very-low density lipoprotein

### BRIEF DESCRIPTION OF THE DRAWINGS

In the following the invention will be described in greater detail by means of preferred embodiments with reference to the attached drawings, in which

FIG. 1 shows mean metabolite levels within each cluster from an analytical platform, shown separately for patients with NAFLD (NAFLD+) and without NAFLD (NAFLD−) in the estimation cohort. Statistical comparison performed by two-sided t test. Error marks show standard error of the mean (*p<0.05, **p<0.01, ***p<0.001);

FIG. 2 shows the relationship between liver fat content and the selected representative metabolites from the clusters which are significantly altered in NAFLD. Correlation coefficients (Spearman rank correlation) and their significance are given. The regression line is drawn as a guide;

FIG. 3 illustrates ROC curves of a liver fat score derived from an analytical platform for molecular lipids (black curves). The diagnostic performance is shown separately for estimation sample, based on which the NAFLD equations were derived, and for the validation sample where the equation was independently tested. Also shown are the ROC curve of the reference model¹ (grey curve). Area under ROC curve (AUC) and corresponding 95% confidence intervals are given. Optimal cut-off points corresponding to the maximum sum of sensitivity and specificity, 95% sensitivity, and 95% specificity are marked in the ROC curves, and corresponding numerical values of the cut-off value along with the specificity and sensitivity are provided;

FIG. 4 illustrates the relationship between measured liver and the predicted liver fat from the model including three lipids, TG(16:0/16:0/16:0), PC(O-24:1/20:4) and PC(18:1/22:6). The shaded circles denote the male subjects and the white circles denote the female subjects. Correlation coefficient and its significance for all subjects are given. Also shown below are the ROC curve for NAFLD diagnosis (estimation and validation series combined, black curve), based on predicted liver fat, and the ROC curve of the reference model¹ (grey curve). AUC and corresponding 95% confidence intervals are given. Optimal cut-off points corresponding to the maximum sum of sensitivity and specificity, 95% sensitivity, and 95% specificity are marked in the ROC curve.

### DETAILED DESCRIPTION OF THE INVENTION

The present invention is based on the idea of determining certain molecular lipids from a subject's blood sample, for example from serum or plasma sample, and based on the amounts of the determined lipids determining the amount of liver fat and/or diagnosing NAFLD in the subject. More specifically the subjects with elevated liver fat amount and NAFLD are characterized by elevated triglycerides with low carbon number and double bond content in the blood sample. Lysophosphatidylcholines, ether phospholipids, sphingomyelins and PUFA-containing phospholipids are diminished in the blood samples of subjects with an elevated liver fat amount and NAFLD.

The method of the present invention can be further used for monitoring the subject's response to the treatment of NAFLD or to the treatment of lowering of the liver fat amount in the subject.

The method of the present invention can be further used for monitoring the subject's response to the prevention of NAFLD or to the prevention of elevation the liver fat amount in the subject.

Elevated liver fat amount and NAFLD both precede and predict type 2 diabetes, the metabolic syndrome, and/or cardiovascular disease, and thus the method of the present invention can be used further in determination of the risk of developing or in determination of early warning signs of these conditions. By determining elevated liver fat amount or diagnosing NAFLD it can be concluded that the risk for type 2 diabetes, the metabolic syndrome, and/or cardiovascular disease has increased in a subject. Thus, elevated liver fat amounts and/or NAFLD can be utilized as predictive markers for type 2 diabetes, the metabolic syndrome, and/or cardiovascular disease.

It has now surprisingly been found out that when the determined concentration in a subject's blood sample of at least one molecular lipid selected from group A consisting of TG(16:0/16:0/18:1), TG(16:0/18:0/18:1), TG(16:0/16:0/16:0) and TG(16:0/18:1/18:1) is increased when compared to the normal mean concentration of respective molecular lipid in healthy individuals without NAFLD, this increased concentration of at least one determined molecular lipid of group A correlates with an increased amount of liver fat of the subject in question. It has also been surprisingly found out that when the determined concentration in a subject's blood sample of at least one molecular lipid selected from group B consisting of lysoPC(16:0), lysoPC(18:0), SM(d18:0/18:0), SM(d18:1/24:1), SM(d18:1/16:0), PC(34:2), PC(18:1/22:6), PC(O-24:1/20:4), PC(34:1e), PC(34:2p), PE(38:2) and PE(36:2), is decreased when compared to the normal mean concentration of respective molecular lipid in healthy individuals without NAFLD, this decreased concentration of at least one determined molecular lipid of group B correlates with an increased amount of liver fat of the subject in question.

The molecular lipids in group A are the following: TG(16:0/16:0/18:1) or dihexadecanoic acid triglyceride, TG(16:0/18:0/18:1) or 1,2-di-(9Z-hexadecenoyl)-3-octadecanoyl-sn-glycerol, TG(16:0/16:0/16:0) or 1,2,3-trihexadecanoyl-glycerol and TG(16:0/18:1/18:1) or 1-hexadecanoyl-2,3-di-(9Z-octadecenoyl)-sn-glycerol.

The molecular lipids in group B are the following: lysoPC(16:0) or 1-palmitoyl-2-hydroxy-sn-glycero-3-phosphocholine, lysoPC(18:0) or 1-octadecanoyl-sn-glycero-3-phosphocholine, SM(d18:0/18:0) or N-(octadecanoyl)-sphinganine-1-phosphocholine, SM(d18:1/24:1) or N-(15Z-tetracosenoyl)-sphing-4-enine-1-phosphocholine, SM(d18:1/16:0) or N-(hexadecanoyl)-sphing-4-enine-1-phosphocholine, PC(18:1/22:6) or 1-(11Z-octadecenoyl)-2-(4Z,7Z,10Z,13Z,16Z,19Z-docosahexaenoyl)-sn-glycero-3-phosphocholine, PC(O-24:1/20:4) or 1-(15Z-tetracosenoyl)-2-(5Z,8Z,11Z,14Z-eicosatetraenoyl)-glycero-3-phosphocholine, PC(34:2), PC(34:1e), PC(34:2p), PE(38:2) and PE(36:2).

The normal mean concentration of each molecular lipid with which the respective determined concentration of the molecular lipid is compared with is determined as the mean concentration in age-matched healthy individuals without NAFLD. The step of comparing concentrations of molecular lipids includes performing statistical analysis. In a preferred embodiment of the invention, the step of comparing any concentrations of molecular lipids includes performing parametric (e.g., t-test, ANOVA) or non-parametric (e.g., Wilcoxon test) statistic.

In the present invention any combination of the concentrations of molecular lipids of group A and/or group B can be determined.

According to one aspect of the present invention the concentrations of PC(O-24:1/20:4), PC(18:1/22:6) and TG(16:0/16:0/16:0) are the determined for determining the liver fat amount and/or NAFLD.

According to another aspect of the present invention the concentrations of PC(18:1/22:6), PC(O-24:1/20:4) and TG(16:0/18:0/18:1) are determined for determining the liver fat amount and/or NAFLD.

In a preferred embodiment of the invention concentrations of molecular lipids are determined by mass spectrometric methods, methods such as Ultra Performance Liquid Chromatography coupled to Mass Spectrometry (UPLC-MS) or by mass spectrometry alone.

Typically the blood sample in the present invention is serum or plasma sample. According to a preferred embodiment of the present invention the plasma sample is citrate plasma or heparin plasma. According to another preferred embodiment of the present invention the serum sample is citrate serum or heparin serum. Taking a blood sample of a subject is part of normal clinical practice. The blood sample can be taken in connection with e.g. measuring the cholesterol levels of the subject. The collected blood sample can be prepared and serum or plasma can be separated with techniques well known for a person skilled in the art.

According to an embodiment of the present invention the subject is a human.

According to one aspect, the method of the present invention can be used for diagnosing non-alcoholic fatty liver disease, wherein an abnormal level of at least one molecular lipid of group A and/or an abnormal level of at least one molecular lipid of group B indicates non-alcoholic fatty liver disease.

An abnormal level of concentration of a molecular lipid is defined to be at least 20% higher than or at least 20% lower than the normal level of concentration, depending of the molecular lipid in question. The normal level of concentration is the mean concentration in age-matched healthy individuals without NAFLD. In other words, if the determined concentration in a subject's blood sample of a molecular lipid selected from group A is at least 20% higher than the normal mean concentration of the respective molecular lipid, this is an abnormal level. If the determined concentration in a subject's blood sample of a molecular lipid selected from group B is at least 20% lower than the normal mean concentration of the respective molecular lipid this is an abnormal level. NAFLD is defined as LFAT>=5.56% of liver tissue weight. This means that when a subject's liver fat percentage is 5.56% or higher of liver tissue weight the subject is diagnosed to have non-alcoholic fatty liver disease.

If the determined concentration in a subject's blood sample of at least one molecular lipid selected from group A consisting of TG(16:0/16:0/18:1), TG(16:0/18:0/18:1), TG(16:0/16:0/16:0) and TG(16:0/18:1/18:1) is increased when compared to the normal mean concentration of respective molecular lipid, this increased concentration of at least one determined molecular lipid of group A correlates with the presence of NAFLD in the subject in question. If the determined concentration in a subject's blood sample of at least one molecular lipid selected from group B consisting of lysoPC(16:0), lysoPC(18:0), SM(d18:0/18:0), SM(d18:1/24:1), SM(d18:1/16:0), PC(34:2), PC(18:1/22:6), PC(O-24:1/20:4), PC(34:1e), PC(34:2p), PE(38:2) and PE(36:2), is decreased when compared to the normal mean concentration of respective molecular lipid, this decreased concentration of at least one determined molecular lipid of group B correlates with the presence of NAFLD in the subject in question.

According to one aspect of the present invention the method of the present invention can be used for monitoring the subject's response to treatment or prevention of non-alcoholic fatty liver disease. The amount of liver fat in the subject is determined with the method of the present invention the obtained value is compared with a previously determined result or with a normal value. The comparison indicates the direction of the development of the non-alcoholic fatty liver disease.

The method of the present invention is especially suitable for subjects having high risk of developing NAFLD, such as subjects who are overweight, have type 2 diabetes, insulin resistance or metabolic syndrome or are at the risk of developing type 2 diabetes, insulin resistance or metabolic syndrome. The method of the present invention is also suitable for subjects having a risk of developing cardio-vascular disease. A laboratory test to non-invasively diagnosing NAFLD and determining liver fat content may be useful in clinical practice both for hepatologists and diabetologists. Prediction of liver fat using a serum or plasma sample takes less time than assessment of the parameters included in the reference method (metabolic syndrome, type 2 diabetes, fasting insulin, and serum ALT and AST concentrations). Knowledge of the amount of fat in the liver may help in selection of patients who should undergo a liver biopsy as liver fat is one predictor of non-alcoholic steatohepatitis (NASH), although prospective data with repeated liver biopsies are needed to examine this possibility. NAFLD also predicts the metabolic syndrome and type 2 diabetes. In patients with type 2 diabetes, the amount of exogenous insulin required to control hepatic glucose production is directly proportional to liver fat, independent of obesity and other factor. Such patients will particularly benefit from interventions effectively reducing liver fat such as weight loss or pioglitazone. A laboratory test measuring the currently described molecular lipids may also be helpful in non-invasively and simply following the response to such therapy.

The concentration of at least one molecular lipid in the blood sample obtained from the subject can be determined with any suitable methods known to a person skilled in the art. According to one embodiment of the present invention the concentration of the molecular lipid is determined by Ultra Performance Liquid Chromatography coupled to Mass Spectrometry (UPLC-MS). For example Nygren et al.² have described a UPLC-MS-based global lipidomics platform, which can be used for determining the concentrations of lipids in accordance with the present invention.

According to one embodiment of the present invention the amount of liver fat in a subject can be determined by providing a blood sample from said subject, determining the concentration of at least one molecular lipid in the blood sample, wherein the molecular lipid is selected from a group A consisting of TG(16:0/16:0/18:1), TG(16:0/18:0/18:1), TG(16:0/16:0/16:0) and TG(16:0/18:1/18:1), and/or a group B consisting of lysoPC(16:0), lysoPC(18:0), SM(d18:0/18:0), SM(d18:1/24:1), SM(d18:1/16:0), PC(34:2), PC(18:1/22:6), PC(O-24:1/20:4), PC(34:1e), PC(34:2p), PE(38:2) and PE(36:2), and the amount of liver fat in the subject is calculated using the following equation (I)

log₁₀(LFAT)=C+a₁[A₁]+a₂[A₂]+a₃[A₃]+ . . . +aₙ[Aₙ]−b₁[B₁]−b₂[B₂]−b₃[B₃]− . . . −bₙ[Bₙ] (I)

wherein

LFAT=amount of liver fat, percentage (%)

C=constant, real number

a₁, a₂, a₃, . . . aₙ=specific coefficient for each molecular lipid of group A, aₙ>0

[A₁], [A₂], [A₃] . . . [Aₙ]=concentration of molecular lipid of group A, μmol/l

b₁, b₂, b₃, . . . bₙ=specific coefficient for each molecular lipid of group B, bₙ>0

[B₁], [B₂], [B₃] . . . [Bₙ]=concentration of molecular lipid of group B, μmol/l

n=number of molecular lipids included in the LFAT equation.

According to one embodiment of the present invention the amount of liver fat in a subject is determined by providing a blood sample from said subject, determining the concentrations of TG(16:0/16:0/16:0), PC(O-24:1/20:4) and PC(18:1/22:6) in said blood sample, calculating the amount of liver fat by the following equation (II)

log₁₀(LFAT)=0.0313929−0.5749506*log([PC(O-24:1/20:4)]−0.5368731*log([PC(18:1/22:6)]+0.08098008*log([TG(16:0/16:0/16:0)]) (II)

wherein
[PC(O-24:1/20:4)], [PC(18:1/22:6)] and [TG(16:0/16:0/16:0)] are the determined concentrations of the respective molecular lipids, μmol/l.

According to one embodiment of the present invention it is possible to diagnose NAFLD from a blood sample obtained from the subject by the following equation (III)

NAFLD(score)=1/(1+e^{-Z}),

wherein Z=1.60236−2.12050×[PC(18:1/22:6)]−0.60689×[PC(O-24:1/20:4)]+0.06931×[TG(16:0/18:0/18:1)] (III)

and wherein
[PC(18:1/22:6)], [PC(O-24:1/20:4)] and [TG(16:0/18:0/18:1)] are the concentrations of the respective molecular lipids, μmol/l.

NAFLD is diagnosed in a subject if NAFLD(score)>0.463.

In the present invention a small set of metabolites was found for which it was feasible to develop a cheap and rapid mass spectrometry based assay which can be performed in a typical clinical chemistry laboratory. Another advantage of serum or plasma metabolic signature to determine liver fat over the liver fat equation which includes multiple biochemical and diagnostic variables¹ is that it, (1) directly reflects the changes in lipid molecular composition of the liver and (2) may also reflect the molecular changes associated with pathogenic mechanisms associated with liver fat such as development of NASH²⁵.

### EXAMPLES

### Methods

### Study Subjects

All subjects were recruited for metabolic studies at the University of Helsinki (Finland) and University of Turku (Finland) mainly by newspaper advertisements, by contacting occupational health services, or among subjects referred to the Department of Gastroenterology because of chronically elevated serum transaminase concentrations using the following inclusion criteria: (1) age 18 to 75 years; (2) no known acute or chronic disease except for obesity or type 2 diabetes based on medical history, physical examination and standard laboratory tests (blood counts, serum creatinine, thyroid-stimulating hormone, electrolyte concentrations) and ECG; (3) alcohol consumption less than 20 g per day. Hepatitis B and C serology, transferrin saturation, anti-smooth muscle antibodies, anti-nuclear antibodies, and anti-mitochondrial antibodies were measured in all patients referred to the gastroenterologist because of chronically elevated liver function tests.

Exclusion criteria included use of thiazolidinedione, and pregnancy. Elevated liver enzymes (serum ALT or AST) were not exclusion criteria. The third cohort was sampled at the Antwerp University Hospital (Belgium), and enrolled patients presenting at the obesity clinic for a problem of overweight. The design and enrollment criteria for this study have been previously reported⁴.

### Study Design

An unthawed blood sample was available from all subjects. The blood samples included were citrate plasma (78% of the samples) as well as heparin plasma and serum samples (16% and 7%, respectively). The subjects were randomly divided into estimation (n=287) and validation (n=392) groups to build and validate the model, respectively. All study subjects (n=679) were used as the second validation group. The validation and estimation groups were comparable with respect to age, gender, BMI, liver fat content, the prevalence of NAFLD, waist circumference, type 2 diabetes, blood pressure, and fasting glucose, triglyceride, HDL cholesterol, and insulin concentrations (Table 1). The prevalence of the metabolic syndrome and liver enzyme concentrations were slightly higher and those of LDL and total serum cholesterol slightly lower in the validation compared to the discovery group (Table 1).

TABLE 1
Characteristics of the estimation and validation groups.

| | Estimation group | | Validation group | | p-value |
|---|---|---|---|---|---|
| N (% men) | 287 | (33) | 392 | (39) | 0.11* |
| Age (y) | 47 ± 11 | | 47 ± 12 | | 0.58 |
| BMI (kg/m²) | 34.7 | (30.6-40.2) | 34.8 | (30.2-41.0) | 0.37 |
| Waist (cm) | 111 ± 17 | | 113 ± 18 | | 0.48 |
| Type 2 diabetes (%) | 21 | | 22 | | 0.50* |
| Metabolic syndrome | 61 | | 69 | | 0.034* |
| (%) | | | | | |
| Liver fat (%) | 4.9 | (1.7-12.4) | 4.8 | (1.0-12.1) | 0.48 |
| NAFLD (%) | 45 | | 45 | | 0.94* |
| fP-glucose (mmol/l) | 5.9 ± 2.0 | | 6.0 ± 1.8 | | 0.28 |
| fS-triglycerides | 1.40 | (1.00-2.00) | 1.40 | (1.00-1.99) | 0.98 |
| (mmol/l) | | | | | |
| fS-HDL cholesterol | 1.25 | (1.06-1.57) | 1.21 | (1.04-1.51) | 0.33 |
| (mmol/l) | | | | | |
| fS-LDL cholesterol | 3.01 ± 0.93 | | 2.83 ± 0.90 | | 0.012 |
| (mmol/l) | | | | | |
| Total serum | 5.1 ± 1.1 | | 4.9 ± 1.0 | | 0.012 |
| cholesterol | | | | | |
| (mmol/l) | | | | | |
| Systolic BP | 133 ± 17 | | 134 ± 17 | | 0.28 |
| (mmHg) | | | | | |
| Diastolic BP | 83 ± 11 | | 85 ± 11 | | 0.062 |
| (mmHg) | | | | | |
| fS-insulin (mU/l) | 12 | (7-17) | 12 | (7-17) | 0.55 |
| S-ALT (U/l) | 32 | (23-48) | 36 | (25-54) | 0.010 |
| S-AST (U/l) | 28 | (22-37) | 30 | (24-41) | 0.005 |
| S-GGT (U/l) | 27 | (19-42) | 32 | (21-57) | 0.006 |

| | | | |
|---|---|---|---|
| Use of statins (%) | 14 | 17 | 0.29* |
| Use of BP | 29 | 36 | 0.047* |
| medications (%) | | | |
| Use of | 9 | 17 | 0.003* |
| antihyperglycemic | | | |
| medication (%) | | | |

Data are shown as mean ± SD or median (interquartile range).
*Chi-square test.

### Liver Fat Content

In 369 subjects (54%), liver fat content was measured using ¹H-MRS as previously described. This measurement has been validated against histologically determined lipid content⁵ and against estimates of fatty infiltration by computed tomography⁶ and MRI⁷. The reproducibility of repeated measurements of liver fat in non-diabetic subjects as determined on two separate occasions in our laboratory is 11%⁸.

In 315 subjects (46%), liver fat was measured using a liver biopsy. The fat content of the liver biopsy specimens (% of hepatocytes with macrovesicular steatosis) was determined by an experienced liver pathologist in a blinded fashion in all subjects based on a hematoxylin-eosin stain. The % of macrovesicular steatosis was converted to liver fat % corresponding to liver fat content measured by ¹H-MRS as previously described⁵.

NAFLD was defined as liver fat ≧55.6 mg triglyceride/g liver tissue or ≧5.56% of liver tissue weight⁹.

### Lipidomic Analysis

Global lipidomics platform based on Ultra Performance Liquid Chromatography coupled to Mass Spectrometry (UPLC-MS) was applied to cover molecular lipids such as phospholipids, sphingolipids, and neutral lipids². Raw data from UPLC-MS analyses were processed using MZmine 2¹⁰ software. The final dataset from each platform consisted of a list of lipid peaks (identified or unidentified) and their levels, calculated using the platform-specific methods, across all samples. All peaks were included in the data analyses, including the unidentified ones. The inclusion of complete data as obtained from the platform best represents the global lipidome.

### Other Analytical Procedures and Measurements

Body weight was recorded to the nearest 0.1 kg using a calibrated weighting scale (Soehnle, Monilaite-Dayton, Finland) with subjects standing barefoot and wearing light indoor clothing. Waist circumference was measured midway between spina iliaca superior and the lower rib margin¹¹. Body height was recorded to the nearest 0.5 centimeter using a ruler attached to the scale. Plasma glucose, serum free insulin, fS-LDL cholesterol, total serum cholesterol, fS-HDL cholesterol, fS-triglyceride, fS-AST, fS-ALT, and fS-GGT concentrations were measured as previously described¹². Blood pressure was measured in the sitting position after 10 to 15 minutes of rest using a random-zero sphygmomanometer (Erka, Germany). In the Belgium cohort measurements were performed as previously described⁴. The metabolic syndrome was defined according to criteria of the International Diabetes Federation.

### Multivariate Statistical Analysis of Metabolomics Data

The data were rescaled into zero mean and unit variance to obtain metabolite profiles comparable to each other for clustering. Bayesian model-based clustering was applied on the scaled data to group lipids with similar profiles across all samples. The analyses were performed using MCLUST¹³ method, implemented in R¹⁴. In MCLUST the observed data are viewed as a mixture of several clusters and each cluster comes from a unique probability density function. The number of clusters in the mixture, together with the cluster-specific parameters that constrain the probability distributions, will define a model which can then be compared to others. The clustering process selects the optimal model and determines the data partition accordingly. The number of clusters ranging from 4 to 15 and all available model families were considered in our study. Models were compared using the Bayesian information criterion (BIC) which is an approximation of the marginal likelihood. The best model is the one which gives the largest marginal likelihood of data, i.e. the highest BIC value.

### Diagnostic Model

In order to perform variable selection we developed a new algorithm based on the methodology of artificial life^{15, 16}. In summary, the algorithm performs a heuristic search for combinations of variables that could predict a specific output variable (either dichotomous or real valued), by evaluating a population of thousands of evolving models, in which better models evolve by combining with each other to produce next generations of models. The simulation is continued until the variables selected in the models do not change further. We used logistic regression¹⁷ for dichotomous output (i.e., classification) and ridge regression¹⁸ for real valued output (i.e., regression) as the modeling method. The areas under ROC curves (AUCs) were compared using the generalized U-statistics¹⁹, implemented in the R package pROC²⁰.

### Results

### Lipidomics in the Estimation Cohort

Using the UPLC-MS analytical platform, a total of 413 molecular lipids were measured in the estimation sample series. Due to a high degree of co-regulation among the metabolites²¹, one cannot assume that all the 1134 measured metabolites are independent. The global metabolome was therefore first surveyed by clustering the data into a subset of clusters using the Bayesian model-based clustering¹³. Lipidomic platform data was decomposed into 9 (LCs). Description of the three clusters which were significantly different between the NAFLD group and healthy controls and representative lipids are shown in Table 2. As expected, the division of clusters to a large degree follows different metabolite functional or structural groups.

TABLE 2
Lipidome cluster descriptions.

| Cluster | Cluster | Cluster | | |
|---|---|---|---|---|
| name | size | description | p-value^{a} | Examples of metabolites^{b} |
| LC2 | 130 | Diverse, | 0.033 | lysoPC(16:0) (↓), |
| | | lysoPCs, | | lysoPC(18:0) (↓), |
| | | SMs, PCs | | SM(d18:0/18:0), |
| | | | | SM(d18:1/24:1), |
| | | | | SM(d18:1/16:0) (↓), |
| | | | | PC(34:2), |
| | | | | PC(18:1/22:6) (↓) |
| LC4 | 82 | Ether PCs, | 0.036 | PC(O-24:1/20:4) (↓), |
| | | PEs | | PC(34:1e) (↓), |
| | | | | PC(34:2p) (↓), |
| | | | | PE(38:2) (↓), |
| | | | | PE(36:2) (↓) |
| LC9 | 32 | SFA and | 7.34E−06 | TG(16:0/18:1/18:1) (↑), |
| | | MUFA | | TG(16:0/16:0/18:1) (↑), |
| | | containing | | TG(16:0/18:1/20:1) (↑) |
| | | TGs | | |

^{a}2-sided t test (NAFLD+ vs. NAFLD− groups in the estimation cohort).
^{b}(↑) and (↓) marks significant up- and down-regulation, respectively, for individual listed metabolites (NAFLD+ vs. NAFLD− groups in the estimation cohort; 2-sided t test).
Abbreviations:
AA, arachidonic acid;
DHA, docosahexanoic acid;
EPA, eicosapentanoic acid;
lysoPC, lysophosphatidylcholine;
PC, phosphatidylcholine.

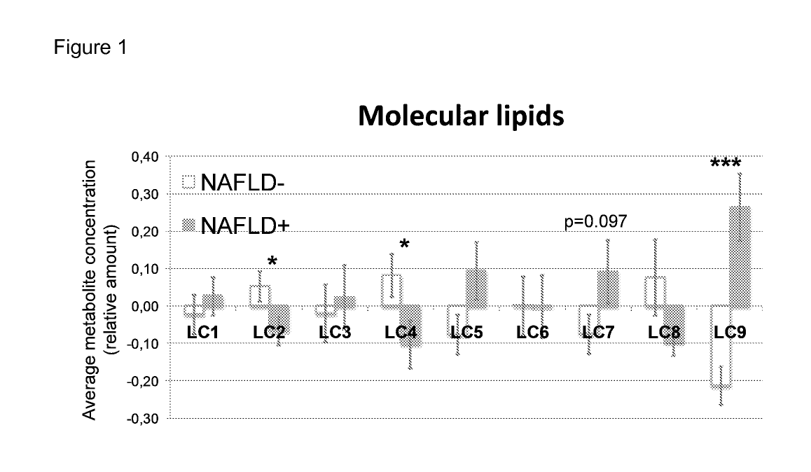

As shown in FIG. 1, several of the clusters had different average metabolite profiles when comparing the subjects with NAFLD and those with normal liver fat (NAFLD+ and NAFLD− groups, respectively). Specifically, there was an overall trend in NAFLD towards increased concentrations of triglycerides containing saturated fatty acid (SFA) and monounsaturated fatty acid (MUFA) (lipid cluster LC9), as well as lower levels of lysophosphatidylcholines (lysoPC) from LC2 and ether lipids (LC4).

### Diagnosis of NAFLD

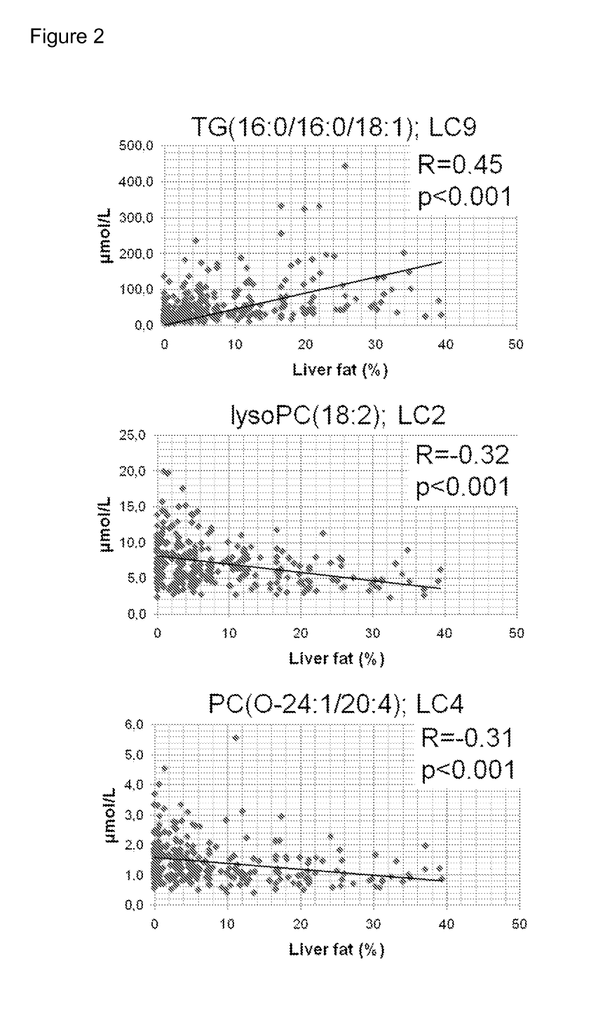

Associations of specific lipid concentrations with the amount of liver fat (FIG. 2) show that blood molecular lipids are predictive of liver fat.

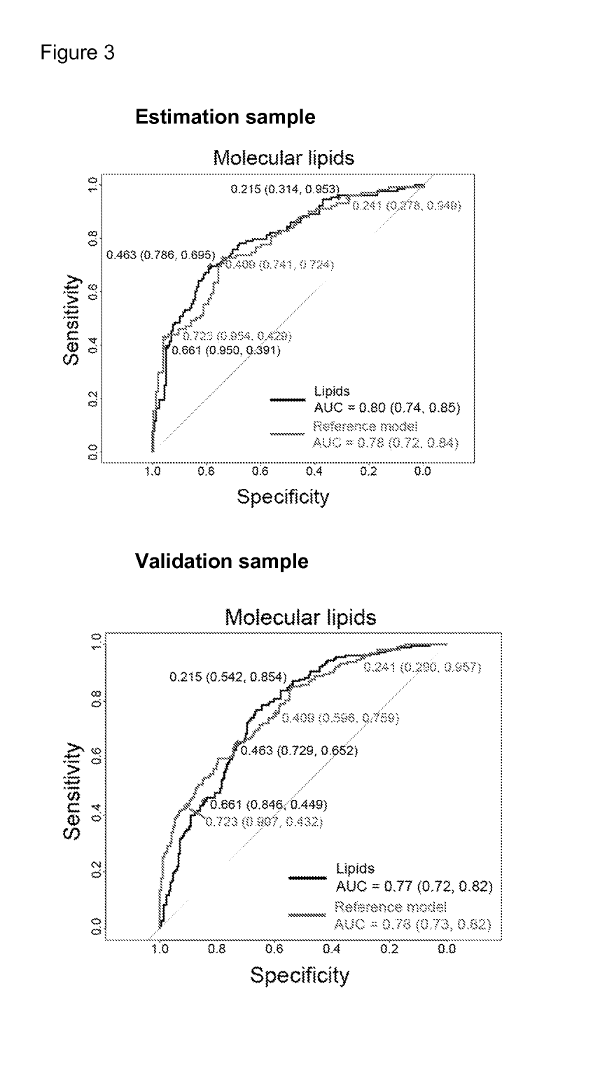

Based on lipidomics in the estimation series, a model using evolutionary algorithm as described in Methods was derived based on UPLC-MS data (FIG. 3). The results were compared with the reference model derived from selected clinical variables¹. Three molecular lipids were selected for the NAFLD equation based on lipidomics data, TG(16:0/18:0/18:1), PC(18:1/22:6), PC(O-24:1/20:4):

NAFLD(score)=1/(1+e^{-Z}),

wherein Z=1.60236−2.12050×[PC(18:1/22:6)]−0.60689×[PC(O-24:1/20:4)]+0.06931×[TG(16:0/18:0/18:1)]

In the estimation series, the lipid-derived model was comparable to the reference model (no significant difference between the ROC curves).

The so-derived model was independently tested in the validation sample series. Lipidomics was applied using the same analytical method as in the estimation sample series. The lipid derived biomarker showed good generalization in the prediction of the validation series subjects (FIG. 3) with no significant difference in AUC compared to the reference model. We have found the optimal cut-off point for classification using Youden's index on the estimation series ROC curve. For the lipid-derived diagnostic model, the optimal cut-off (0.463) has resulted in a diagnostic test with 69.5% sensitivity and 78.6% specificity (i.e. NAFLD is diagnosed for NAFLD(score)>0.463). When applied this test to the validation series, the sensitivity and specificity were 65.2% and 72.9% respectively. The reference model in combination with its optimal cut-off point attained a test with 72.4% sensitivity and 74.1% specificity in the estimation series.

Since the lipids included in the NAFLD model include an abundant triglyceride together with two PUFA-containing lipids which are both negatively associated with liver fat (Table 2) and are both common constituents of HDL²²⁻²⁴, we also tested if TG(16:0/18:0/18:1) together with HDL cholesterol would predict NAFLD. The model was developed from the estimation series using the same methodology as above. It performed reasonably well, with AUC=0.74 (95% CI: 0.69-0.80) in the estimation series and AUC=0.71 (95% CI: 0.67-0.77) in the validation series. However, the AUC was significantly lower both in the estimation and validation sample as compared to both the lipidomic signature as well as the reference model.

Determination of Liver Fat Amount from Serum Lipidomic Profile

Using the lipidomics data from the estimation sample series we derived a model to determine the liver fat content, applying the artificial life algorithm and ridge regression similarly as for the NAFLD model. Based on the data in the estimation series, the following model for liver fat content was derived using concentration information from three model-selected molecular lipids, PC(O-24:1/20:4), PC(18:1/22:6) and TG(48:0):

log₁₀(LFAT)=0.0313929−0.5749506×log([PC(O-24:1/20:4)])−0.5368731×log([PC(18:1/22:6)])+0.8098008×log([TG(48:0)]).

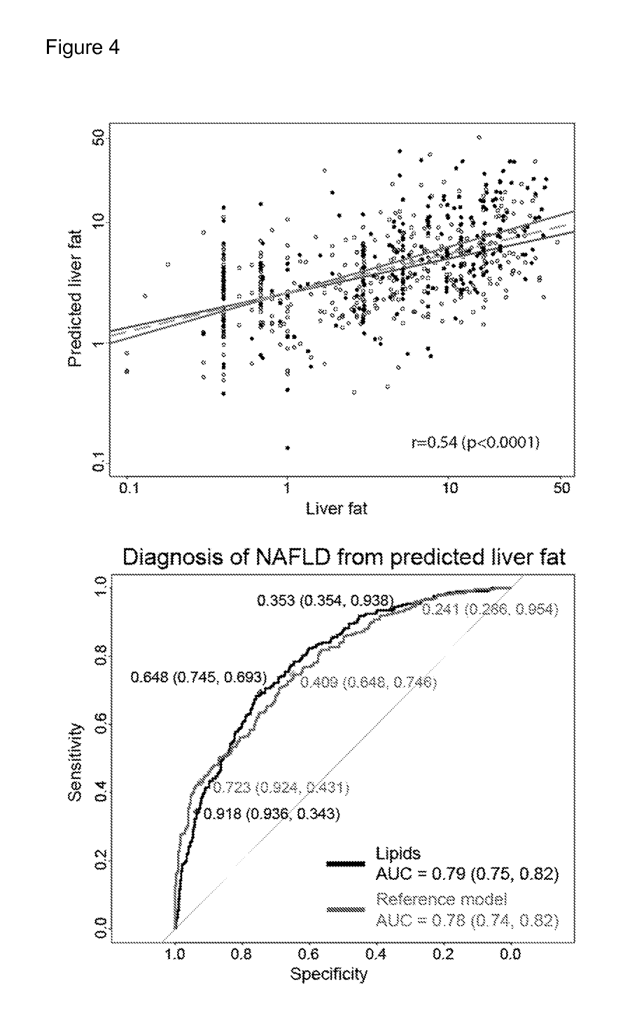

FIG. 4 shows the model performance when applied to the estimation and validation data taken together. We also applied the liver fat model above for the diagnosis of NAFLD. FIG. 4 shows the ROC curves of the diagnostic performance of the model. Interestingly, this model not only shows better performance than the original NAFLD diagnostic model (FIG. 3), but also performs at least as good as the reference model. However, it is not significantly better than the reference model. The optimal cut-off point as determined by the Youden's index resulted in a test with 69.5% sensitivity and 75.5% specificity in the estimation series. When applied to the validation series, the sensitivity and specificity are 69.1% and 73.8%, respectively. Moreover, when applied to the estimation and validation series samples together, the sensitivity and specificity are 69.3% and 74.5%, respectively. As a comparison, the reference model performs with 74.6% sensitivity and 64.8% specificity when applied to estimation and validation series together.

The association of the lipid signature and liver fat content is clearly stronger as for fasting insulin and AST, respectively. In order to evaluate if addition of other biochemical or clinical data could improve the prediction of liver fat, we also trained a model by adding fasting insulin as an additional term to the three lipid markers, using artificial life ridge regression algorithm as above. However, no improvement in prediction of liver fat was observed from a combined model including three lipids and fasting insulin. Using the same methodology, we also developed a combined model using available diagnostic, clinical and metabolomics data by imposing that the model contains at least one variable from each of the three categories: (1) diagnostic and clinical, (2) molecular lipids acquired by UPLC-MS and (3) GC×GC-TOFMS derived small polar metabolites. The best model contained diagnosis of diabetes mellitus, amino acid lysine (negatively associated with liver fat) and triglyceride TG(16:0/18:0/18:1). However, the combined model did not improve the prediction of liver fat or diagnosis of NAFLD as compared to the lipid-based model or the reference model.

It will be obvious to a person skilled in the art that, as the technology advances, the inventive concept can be implemented in various ways. The invention and its embodiments are not limited to the examples described above but may vary within the scope of the claims.

### LIST OF REFERENCES

1. Kotronen A, Peltonen M, Hakkarainen A, Sevastianova K, Bergholm R, Johansson L M, Lundbom N, Rissanen A, Ridderstråle M, Groop L, Orho-Melander M, Yki-Järvinen H. Prediction of non-alcoholic fatty liver disease and liver fat using metabolic and genetic factors. Gastroenterology 2009; 137:865-872.

2. Nygren H, Seppanen-Laakso T, Castillo S, Hyotylainen T, Oresic M. Liquid Chromatography-Mass Spectrometry (LC-MS)-Based Lipidomics for Studies of Body Fluids and Tissues. Methods Mol. Biol. 2011; 708:247-57.

3. Castillo S, Mattila I, Miettinen J, Ore\check{s}i\check{c} M, Hyötyläinen T. Data analysis tool for comprehensive two-dimensional gas chromatography-time of flight mass spectrometry Anal. Chem. 2011; 83:3058-3067.

4. Francque S, Verrijken A, Mertens I, Hubens G, Van Marck E, Pelckmans P, Michielsen P, Van Gaal L. Visceral adiposity and insulin resistance are independent predictors of the presence of non-cirrhotic NAFLD-related portal hypertension. Int. J. Obes. (Lond) 2011; 35:270-8.

5. Kotronen A, Vehkavaara S, Seppala-Lindroos A, Bergholm R, Yki-Jarvinen H. Effect of liver fat on insulin clearance. Am. J. Physiol. Endocrinol. Metab. 2007; 293:E1709-15.

6. Ryysy L, Hakkinen A M, Goto T, Vehkavaara S, Westerbacka J, Halavaara J, Yki-Jarvinen H. Hepatic fat content and insulin action on free fatty acids and glucose metabolism rather than insulin absorption are associated with insulin requirements during insulin therapy in type 2 diabetic patients. Diabetes 2000; 49:749-58.

7. Machann J, Thamer C, Schnoedt B, Stefan N, Haring H U, Claussen C D, Fritsche A, Schick F. Hepatic lipid accumulation in healthy subjects: a comparative study using spectral fat-selective MRI and volume-localized 1H-MR spectroscopy. Magn. Reson. Med. 2006; 55:913-7.

8. Sutinen J, Hakkinen A M, Westerbacka J, Seppala-Lindroos A, Vehkavaara S, Halavaara J, Jarvinen A, Ristola M, Yki-Jarvinen H. Increased fat accumulation in the liver in HIV-infected patients with antiretroviral therapy-associated lipodystrophy. AIDS 2002; 16:2183-93.

9. Szczepaniak L S, Nurenberg P, Leonard D, Browning J D, Reingold J S, Grundy S, Hobbs H H, Dobbins R L. Magnetic resonance spectroscopy to measure hepatic triglyceride content: prevalence of hepatic steatosis in the general population. Am. J. Physiol. Endocrinol. Metab. 2005; 288:E462-8.

10. Pluskal T, Castillo S, Villar-Briones A, Oresic M. MZmine 2: Modular framework for processing, visualizing, and analyzing mass spectrometry-based molecular profile data. BMC Bioinformatics 2010; 11:395.

11. Marti B, Tuomilehto J, Salomaa V, Kartovaara L, Korhonen H J, Pietinen P. Body fat distribution in the Finnish population: environmental determinants and predictive power for cardiovascular risk factor levels. J. Epidemiol. Community Health 1991; 45:131-7.

12. Kotronen A, J. W, Bergholm R, Pietiläinen K H, Yki-Järvinen H. Liver fat in the metabolic syndrome. J. Clin. Endocrinol. Metab. 2007; 92:3490-3497.

13. Fraley C, Raftery A E. Model-based methods of classification: Using the mclust software in chemometrics. J. Stat. Soft. 2007; 18:1-13.

14. The R Project for Statistical Computing. Volume 2011.

15. Mitchell M, Forrest S. Genetic algorithms and artificial life. Artificial Life 1994; 1:267-289.

16. Maes P. Modeling adaptive autonomous agents. Artificial Life 1993; 1:135-162.

17. McCullogh P, Nelder J A. Generalized linear models. Chapman & Hall/CRC, 1989.

18. Hoerl A E, Kennard R W. Ridge regression: Biased estimation for nonorthogonal problems. Technometrics 1970; 12:55-67.

19. DeLong E R, DeLong D M, Clarke-Pearson D L. Comparing the areas under two or more correlated receiver operating characteristic curves: a nonparametric approach. Biometrics 1988; 44:837-45.

20. Robin X, Turck N, Hainard A, Tibet N, Lisacek F, Sanchez J C, Muller M. pROC: an open-source package for R and S+ to analyze and compare ROC curves. BMC Bioinformatics 2011; 12:77.

21. Steuer R, Kurths J, Fiehn O, Weckwerth W. Observing and interpreting correlations in metabolomic networks. Bioinformatics 2003; 19:1019-1026.

22. Wiesner P, Leidl K, Boettcher A, Schmitz G, Liebisch G. Lipid profiling of FPLC-separated lipoprotein fractions by electrospray ionization tandem mass spectrometry. J. Lipid Res. 2009; 50:574-585.

23. Kotronen A, Velagapudi V R, Yetukuri L, Westerbacka J, Bergholm R, Ekroos K, Makkonen J, Taskinen M R, Oresic M, Yki-Jarvinen H. Serum saturated fatty acids containing triacylglycerols are better markers of insulin resistance than total serum triacylglycerol concentrations. Diabetologia 2009; 52:684-90.

24. Yetukuri L, Söderlund S, Koivuniemi A, Seppänen-Laakso T, Niemelä P S, Hyvönen M, Taskinen M R, Vattulainen I, Jauhiainen M, Oresic M. Composition and lipid spatial distribution of HDL particles in subjects with low and high HDL-cholesterol. J. Lipid Res. 2010; 51:2341-51.

25. Barr J, Vazquez-Chantada M, Alonso C, Perez-Cormenzana M, Mayo R, Galan A, Caballeria J, Martin-Duce A, Tran A, Wagner C, Luka Z, Lu S C, Castro A, Le Marchand-Brustel Y, Martinez-Chantar M L, Veyrie N, Clement K, Tordjman J, Gual P, Mato J M. Liquid chromatography-mass spectrometry-based parallel metabolic profiling of human and mouse model serum reveals putative biomarkers associated with the progression of nonalcoholic fatty liver disease. J Proteome Res 2010; 9:4501-12.

1. A method for determining a subject's liver fat amount comprising
a) providing a blood sample from said subject,
b) determining the concentration of at least one molecular lipid in the blood sample, wherein the molecular lipid is selected from
i) group A consisting of TG(16:0/16:0/18:1), TG(16:0/18:0/18:1), TG(16:0/16:0/16:0) and TG(16:0/18:1/18:1), and/or
ii) group B consisting of lysoPC(16:0), lysoPC(18:0), SM(d18:0/18:0), SM(d18:1/24:1), SM(d18:1/16:0), PC(34:2), PC(18:1/22:6), PC(O-24:1/20:4), PC(34:1e), PC(34:2p), PE(38:2) and PE(36:2),
wherein an increased concentration of at least one molecular lipid of group A compared to the normal mean concentration of respective molecular lipid, and/or
a decreased concentration of at least one molecular lipid of group B compared to the normal mean concentration of respective molecular lipid correlates with the liver fat amount in said subject.

2. The method according to claim 1, wherein the concentrations of any combination of molecular lipids of group A and/or group B is determined.

3. The method according to claim 1 or 2 wherein the concentrations of PC(O-24:1/20:4), PC(18:1/22:6) and TG(16:0/16:0/16:0) are determined.

4. The method according to claim 1 or 2 wherein the concentrations of PC(18:1/22:6), PC(O-24:1/20:4) and TG(16:0/18:0/18:1) are determined.

5. The method according to claim 1, wherein the blood sample is a plasma sample or a serum sample.

6. The method according to claim 1, wherein the subject is a human.

7. The method according to claim 1 for use in diagnosing non-alcoholic fatty liver disease (NAFLD).

8. The method according to claim 1 for use in monitoring a subject's response to treatment of non-alcoholic fatty liver disease (NAFLD) or to treatment of lowering of the liver fat amount in the subject.

9. The method according to claim 1 for use in monitoring a subject's response to prevention of non-alcoholic fatty liver disease (NAFLD) or to prevention of elevation of the liver fat amount in the subject.

10. The method according to claim 1 for use in determining the risk of developing or in determining early warning signs of type 2 diabetes, the metabolic syndrome, and/or cardiovascular disease.
